(19) United States
(12) Patent Application Publication
(10) Pub. No.: US 2026/0240275 A1
(43) Pub. Date: Aug. 20, 2026

(54) BODY IMPACT EVALUATION SYSTEM AND WEARABLE MEASUREMENT SYSTEM THEREFOR

(71) Applicant: ECOLE POLYTECHNIQUE FEDERALE DE LAUSANNE (EPFL), Lausanne (CH)

(72) Inventors: Mathieu Pascal FALBRIARD, LAUSANNE (CH); Tom-Alexandre BERTRAND, ST-SULPICE VD (CH); Harshal Arun SONAR, CHAVANNES-RENENS (CH); Christophe GUBERAN, LA PRAZ (CH); Kamiar AMINIAN, LA TOUR-DE-PEILZ (CH)

(21) Appl. No.: 19/160,973

(22) PCT Filed: Feb. 29, 2024
(86) PCT No.: PCT/EP2024/055190

§ 371 Date: Aug. 29, 2025

(30) Foreign Application Priority Data
Mar. 1, 2023 (EP) 23159279.1

(51) Int. Cl. A42B 3/04; A61B 5/00

(52) U.S. Cl. CPC A42B 3/046; A61B 5/4064; A61B 5/6803; A61B 5/7267; A61B 2562/0214; A61B 2562/0247; A61B 2562/168

(57) ABSTRACT

A measurement system for a wearable item comprising a capacitive pressure sensing system (5) including a plurality of capacitive sensors (6) comprising a deformable insulator layer (32a, 32b) and at least first and second electrodes (30a, 30b, 30c) on opposed sides of the deformable insulator layer, the capacitive sensors arranged in a distributed manner over a surface for covering a body part subject to impact. The capacitive sensors comprise first and second deformable insulator layers (32a, 32b) separated by a third electrode (32c), the first insulator layer having an elastic modulus that is different or the same to the elastic modulus of the second insulator layer, the capacitive sensors configured to measure a pressure applied on a major surface of the sensor transverse to the stacking direction of the electrodes and deformable insulator layers.

### FIELD OF THE INVENTION

This invention relates to a body impact evaluation system including a wearable measurement system therefor.

### BACKGROUND OF THE INVENTION

Many sports activities such as ice hockey, American football, cycling, motorsports, and skiing are associated with a risk of traumatic brain injury (TBI). Sports like ice hockey and skiing mainly rely on protective helmet equipment for the prevention of major head injuries. However, during the actual hockey game, up to 40% of TBI-related injuries go unnoticed, and the player doesn't receive the necessary support. Modern sports helmets certified to various international safety standards have virtually eliminated the incidence of cranial fracture and fatal brain injury in contact sports; however, the occurrence of diffuse brain injuries (mild-TBI) is still prevalent and leads to important time loss from the sport.

To mitigate these risks, it is known to provide wearable monitoring devices with embedded electronic instrumentation for the detection of concussion-related impacts. Most widely used systems with helmet-based instrumentation today rely on the estimation of the rotational velocities using gyroscopes and linear acceleration using accelerometers. Head Impact Telemetry (HIT) based systems using only acceleration data have limited performance for measuring angular rotations. More advanced gyroscope and accelerometer-based systems can be mounted on the helmets and provide real-time measurements but are still limited in the accuracy of translating the measured impact acceleration to the force vector applied to a human head. The force vector would help to accurately model the impact effects on the human brain using tools like finite element analysis. Conventional approaches lack an accurate direction of the force vector and area of the applied impact that is crucial to precisely model the impact dynamics.

Recent research efforts have tried using resistive strain sensors in a distributed array manner as a sensing layer to validate the safety performance of ice hockey helmets. The resistive sensors however are limited in resolution and suffer from temperature drift and therefore also require frequent sensor calibrations. An improved approach is provided by the use of porous Polydimethylsiloxane (PDMS) based foam type of sensor that measures the capacitance of the system as opposed to the resistance. Even though capacitive sensing is more repetitive and accurate than resistive technology, it often requires an additional instrumentation circuit close to the sensor to avoid stray capacitance accumulation that otherwise leads to reduced sensitivity.

Specific injury-mechanism hypotheses include linear motions (translation of the head), which can produce intracranial pressure gradients and lead to focal injuries, and angular motions (rotation of the head), which produce shear stresses and tensile strains that may lead to diffuse axonal injuries. As it is currently impractical to measure pressure gradients and brain strains in vivo during sports participation, head impact sensors are used to measure the motion of the head on the assumption that brain strains are in some way proportional to the magnitude of this motion. Although the use of impact sensors is now common, published meta-analyses highlight the large heterogeneity in data collection methods (e.g., acceleration thresholds) and exposure metrics. Also, conventional systems do not allow to accurately analyse impacts that cause mild-TBI in athletes involved in collisions which would help in taking corrective measures.

Studies on repetitive head impacts have shown that a similar impact may have significantly different consequences on a player's brain health. An accurate history of the measurement of recurrent head impacts is crucial to make an informed estimation of the brain damages induced by future impacts, especially after a diagnosed mild-TBI. Accurate measurements of past head injuries of players could be used to recursively re-evaluate the threshold for permissible severity of impact during gameplay.

In wearable gear, it is also important to provide sensing systems that are unobtrusive and economical.

### SUMMARY OF THE INVENTION

An object of the invention is to provide a wearable impact sensing system that provides an accurate measurement of impact parameters that can be integrated in wearable gear such as helmets in an economical manner.

It is advantageous to provide a body impact evaluation system that is light weight and unobtrusive and may be easily conformed for integration in wearable parts such as helmets and shoewear.

A particular object of the invention is to provide an impact evaluation system for helmets, in particular helmets for sporting activities.

It is an object of this invention to provide an impact evaluation system for helmets that allows assessment of brain injury including mild traumatic brain injury in sports persons and to reduce the repercussions thereof.

It is advantageous to provide a wearable impact sensing system that is economical to manufacture.

Objects of this invention have been achieved by providing the wearable impact sensing system according to the independent claims. Dependent claims set forth various advantageous embodiments of the invention.

Disclosed herein according to a first aspect of the invention, is a measurement system for a wearable item comprising a capacitive pressure sensing system including a plurality of capacitive sensors comprising a deformable insulator layer and at least first and second electrodes on opposed sides of the deformable insulator layer, the capacitive sensors arranged in a distributed manner over a surface for covering a body part subject to impact, wherein the capacitive sensors comprise first and second deformable insulator layers separated by a third electrode, the first insulator layer having an elastic modulus that is different to an elastic modulus of the second insulator layer, and/or the first insulator layer having a thickness that is different to a thickness of the second insulator layer, the capacitive sensors configured to measure a pressure applied on a major surface of the sensor transverse to the stacking direction of the electrodes and deformable insulator layers.

In an advantageous embodiment, the deformable insulator layers comprise or consist of a polymer open or closed cell foam material.

In an advantageous embodiment, the electronic control system further comprises an onboard measurement signal analysis module and a memory, the onboard measurement signal analysis module including a machine learning module and an impact analysis module configured to compute at least linear acceleration and inertial angular acceleration on a center of gravity and a center of rotation of a body part.

In an advantageous embodiment, the measurement system comprises a multiplexer connected to the fluidic and/or capacitive pressure sensing system, the multiplexer comprising an analog multiplexer with or without a digital converter to aid in preventing sensor signal from corrupting due to noise arranged between sensing elements of the fluidic and/or capacitive pressure sensing system and separate from the computing system.

In an advantageous embodiment, the measurement system comprises a wireless communications system including any one or more of a Bluetooth communication system, a Wifi communication system, a GSM communication system, or a near field communication system for connection to an external computing unit.

In an advantageous embodiment, the measurement system comprises any of the additional features of the second aspect of the invention described below.

Also disclosed herein, according to a second aspect of the invention, is a measurement system for a wearable item comprising a fluidic pressure sensing system including a plurality of capsule chambers each filled with a fluid, and pressure sensors connected to the capsule chambers configured to measure a variation in volume due to pressure applied on an outer wall of the chamber, the measurement system further comprising an inertial measurement unit (IMU) and an electronic control system receiving measurement signals from the pressure sensors and inertial measurement unit and for processing said signals to compute parameters related to an impact.

In an advantageous embodiment, the chambers are formed within a deformable polymeric material.

In an advantageous embodiment, the said deformable material is selected from a group of materials comprising natural rubber or artificial polymers, deformable air-tight fabric or fibre re-enforced stretchable silicone or natural rubber.

In an advantageous embodiment, the capsule chamber is formed within a deformable capsule separately formed from a support structure of the wearable item, the deformable capsule comprising a capsule outer wall, a plurality of deformable capsules arranged in a distributed manner for covering a body part subject to impact, each deformable capsule connected individually to a pressure sensor or an individual inlet of a common pressure sensor.

In an advantageous embodiment, the capsule chamber comprises a surface area of between 3 cm² to 300 cm², preferably in a range of 10 cm² to 200 cm² over a surface that is substantially orthogonal to an impact direction, and a thickness in said impact direction in a range between 5 and 35 mm, preferably in a range of between 8 and 20 mm.

In an advantageous embodiment, the capsule chambers have a substantially triangular shape.

In an advantageous embodiment, the measurement system further comprises a capacitive sensing system including a plurality of capacitive sensors comprising a deformable insulator layer and at least first and second electrodes on opposed sides of the deformable insulator layer, the capacitive sensors arranged in a distributed manner over a surface for covering a body part subject to impact.

In an advantageous embodiment, the deformable insulator layer comprises or consists of a polymer foam material.

In an advantageous embodiment, the capacitive sensors comprise first and second deformable insulator layers separated by a third electrode.

In an advantageous embodiment, the first insulator layer has an elastic modulus that is different to an elastic modulus of the second insulator layer, and/or the first insulator layer has a thickness that is different to a thickness of the second insulator layer, the capacitive sensors configured to measure a pressure applied on a major surface of the sensor transverse to the stacking direction of the electrodes and deformable insulator layers.

In an advantageous embodiment, the electronic control system comprises a sensor signal processing circuit configured to process both the inputs for the inertial measurement unit and the pressure sensors, and optionally from additional sensors such as the capacitive sensors (6), for generating impact measurement parameters including an amplitude of the impact force and location of the impact force.

In an advantageous embodiment, the electronic control system further comprises an onboard measurement signal analysis module and a memory, the onboard measurement signal analysis module including a machine learning module and an impact analysis module configured to compute at least linear acceleration and inertial angular acceleration on a center of gravity and a center of rotation of a body part.

In an advantageous embodiment, the measurement system comprises a multiplexer connected to the fluidic pressure sensing system, the multiplexer comprising an analog multiplexer arranged between sensing elements of the fluidic pressure sensing system and separate from the computing system.

In an advantageous embodiment, the measurement system further comprises a wireless communications system including any one or more of a Bluetooth communication system, a Wifi communication system, a GSM/LTE communication system, or a near field communication system for connection to an external computing unit.

Also disclosed herein is a wearable item comprising a sensor support structure and the measurement system according to any of the above described aspects and embodiments mounted on or in the sensor support structure.

In an advantageous embodiment, the wearable item is a protective headgear, for instance in the form of a helmet.

In an advantageous embodiment, the sensor support structure is formed of a deformable polymeric self-supporting material, the fluidic pressure sensing system being integrated into the sensor support structure.

In an advantageous embodiment, the fluidic chambers of the measurement system are formed within the sensor support structure.

In an advantageous embodiment, the wearable item is in the form of a helmet, the multiplexer being positioned in a top region of the helmet.

In an advantageous embodiment, the multiplexer is a slave analogue circuit or a micro-controller based digital multiplexer configured to provide sensor data on demand to the microcontroller master.

In an advantageous embodiment, the capacitive sensors of the capacitive pressure sensing system include capacitive sensors positioned in the helmet at least on a front left, front right, back left and back right positions, optionally wherein the measurement system further includes capacitive sensors in a front center and back center position.

In an advantageous embodiment, the chambers of the fluidic pressure sensing system include chambers positioned in the helmet at least on a front left, front right, back left and back right positions.

In an advantageous embodiment, the measurement system further includes chambers in a front center and back center position.

In an advantageous embodiment, the capacitive sensors are positioned in the form of a protective layer within the helmet.

In an advantageous embodiment, the capacitive sensors are arranged between, above, or below the fluidic pressure sensing system.

Further objects and advantageous aspects of the invention will be apparent from the claims, and from the following detailed description and accompanying FIGS.

### BRIEF DESCRIPTION OF THE DRAWINGS

FIG. 1a is a schematic side view of a helmet including elements of an impact sensing system, in particular fluidic pressure sensing system elements according to an embodiment of the invention;

FIG. 1b is a schematic top view of the helmet of FIG. 1a;

FIG. 1c is a schematic side view of a helmet with fluidic pressure sensing system according to a variant;

FIG. 1d is a schematic side view of a support structure of the helmet of FIG. 1c;

FIGS. 2a to 2c are illustrations of deformable capsules of a fluidic pressure sensing system of a body impact evaluation system according to an embodiment of the invention;

FIG. 2d is a schematic cross-sectional side view of the deformable capsule of FIGS. 2a to 2c, connected to a pressure sensor;

FIG. 2e is a graph of measured pressure compared to the applied pressure of a deformable capsule according to FIGS. 2a to 2d;

FIGS. 3a and 3b are views similar to FIGS. 1a and 1b of an embodiment including capacitive sensors;

FIGS. 4a to 4c are schematic representations of a capacitive pressure sensor according to an embodiment of the invention;

FIG. 4d is a graph of measured capacitance versus the applied force on a capacitive sensor according to FIG. 4c;

FIG. 5 is a block diagram illustrating the architecture of a body impact evaluation system according to an embodiment of the invention;

FIGS. 6a and 6b are schematic illustrations of a helmet with an impact measurement system according to an embodiment the invention illustrating measured or calculated parameters relevant for impact evaluation;

FIG. 6c is a simplified process flow diagram illustrating the measurement process of an impact according to an embodiment of the invention;

FIGS. 7a to 7c are schematic views of a helmet illustrating measured and computed parameters and FIG. 7d is a process flow diagram illustrating the steps computed by an algorithm for computation of parameters relevant for the evaluation of an impact according to an embodiment of the invention;

FIGS. 8a to 8c illustrate measured and computed vectors and parameters relevant for impact evaluation according to an embodiment of the invention;

FIG. 9a is a schematic perspective view of a capacitive pressure sensor according to an embodiment of the invention and

FIGS. 9b and 9c are schematic circuit representations of a capacitive sensor according to embodiments of the invention.

### DETAILED DESCRIPTION OF EMBODIMENTS OF THE INVENTION

Referring to the FIGS., a body impact evaluation system comprises a wearable item 2 and a measurement system 3 integrated in the wearable item. According to one of the aspects of the invention, the wearable item is in a helmet, in particular a helmet for sports activities such as ice hockey, American football, boxing, and other sports employing headgear. The body impact evaluation system may however also be implemented in other body protection gear such as body armour in contact sports (waist, knee, chin, wrist and elbow protective gear) and in footwear especially the soles of running shoes.

Protective gear may also comprise body impact evaluation systems in professional activities such as protective gear for workers in the building industry.

The invention is particularly well adapted for use in protective headgear in sporting activities where players are subject to repeated impacts or occasional severe impacts to their head.

According to an aspect of the invention, the body impact evaluation system is in particular adapted for the assessment of traumatic brain injury, including the assessment of mild traumatic brain injury. The results of the assessment of the impacts may serve to determine the severity of an impact in various activities including not only the above-mentioned sports but for instance in motorsports in order to provide alerts for the severity of an impact on a body part of a person. Embodiments of the invention are however particularly well suited for the assessment and recording of repeated impacts in order to assess the possible medical repercussions over time and allow for an accurate diagnosis and possible measures to mitigate health risks to the person.

The wearable item may in particular comprise a sensor support structure in a self-supporting form conforming to a body part in which the wearable item is intended to be placed against. The sensor support structure may for instance be made of a rigid or semi-rigid polymer foam material or be made of a rigid protective shell material such as a thermoplastic or thermosetting polymer with or without reinforcement fibers, such materials and structures per se known for use in protective headgear and body armour.

The body impact evaluation system may further comprise an external base station 100 or communication system 102 configured to communicate with the measurement system 3 of the wearable item for receiving and processing measurement data and optionally for transmitting firmware and/or other software and data to the onboard measurement system 3, for instance for updating the firmware and for remote configuration of the measurement system 3 integrated in the wearable item 2.

The external base station 100 may be provided in various forms, for instance comprising hardware such as a computer tablet, smartphone, comprising either wireless communications systems according to various protocols such as Radio, Bluetooth or Wifi or for connection via the internet for transmission of data between the base station 100 and the measurement system 3 of the wearable item 2.

The measurement system 3 comprises an impact sensing system 4 and an electronic control system 8.

The impact sensing system 4 comprises a fluidic pressure sensing system 5 including a deformable capsule 16 having an outer wall 18 forming a chamber 22 therein filled with a fluid, the chamber 22 connected via an inlet/outlet 24 and a fluidic connection conduit 26 to a pressure sensor 28. Pressure applied on the capsule's outer wall deforms the capsule and increases the fluid pressure within the chamber 22 that is measured by the pressure sensor 28.

In a preferred embodiment, the fluid contained within the deformable capsule 6 is a gas such as air, nitrogen, argon, or carbon dioxide. The deformable capsules may simply be filled with air, however in order to reduce the diffusion of gas through the capsule walls, gases with larger molecular sizes may be used such as nitrogen or carbon dioxide. In variants of the invention, the capsule may also be filled with a liquid. In variants, internal supporting elements or material 20 may be provided within the chamber 22, configured to push the capsule outer walls after deformation back to their original shape prior to application of the external pressure.

The capsule wall may be made of a polymer material, for instance an elastomeric polymer material such as natural rubber or artificial soft polymers like silicone rubbers, stretchable nylon, Thermoplastic Polyurethane (TPU/TPE-U), or woven fabric with airtight sheathing using Silicone or TPU/TPE-U. The capsule outer wall may however be provided in a material that is deformable, for instance such as a thin foil, but that does not have any particularly elastic properties, the elastic restitution of the shape of the capsule being provided by internal elastic support means (not shown) within the capsule chamber 22.

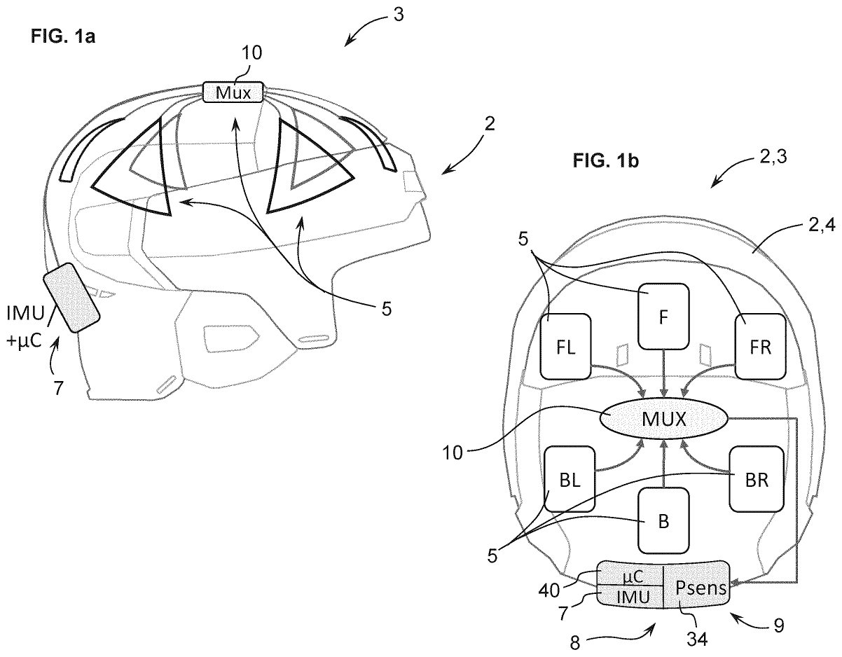

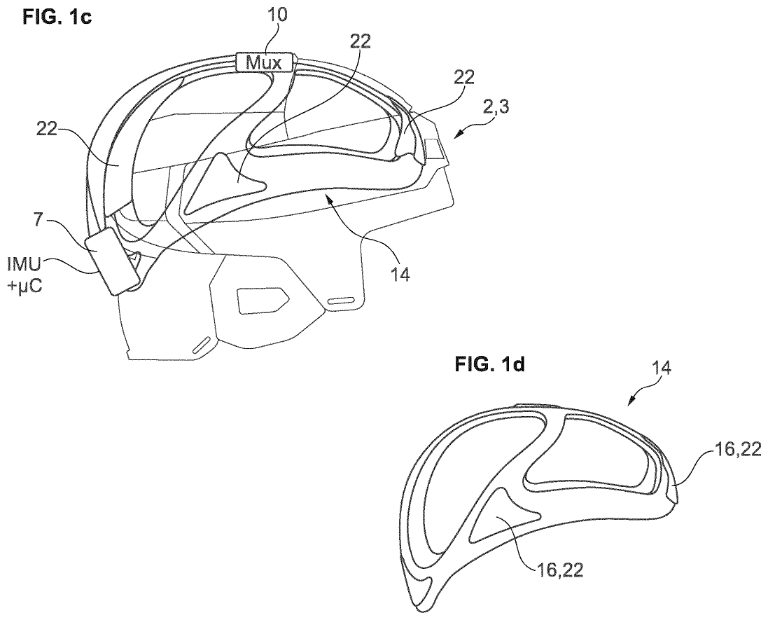

In a variant, the fluidic pressure sensing system 5 may comprise capsule chambers 22 filled with a fluid that are formed directly within a sensor support structure 14 as illustrated for instance in FIGS. 1c and 1d.

In such variant, the capsules are thus directed and formed in the material of the surrounding support structure. The sensor support structure 14 may for instance be made of a single part by additive manufacturing or with various moulding techniques, possibly with a plurality of composite parts that are then welded or bonded together.

The sensor support structure with integrated fluidic capsule chambers may further include fluidic connection conduits therein that channel the fluid from the chamber to a pressure sensor coupled to the sensor support structure 14 for measuring the change in fluid pressure due to deformation of the chamber upon impact or application of an external pressure.

According to an aspect of the invention, the fluidic pressure sensing system comprises a plurality of deformable capsules 16, each connected via its own fluidic connection conduit 26 to a corresponding pressure sensor 28, the individual deformable capsules 16 being assembled or formed in different positions in the wearable item 2. Thus, each deformable capsule allows for providing a measurement of the pressure applied to a certain section of the wearable item 2. For instance, in the illustrated embodiment, the helmet 2 is provided with deformable capsules positioned on the front left (FL) and front right (FR) of the helmet and the back left (BL) and the back right (BR). There may be additional deformable capsules, for instance in the center front (F) and center back (B) and optionally on the center left and center right (not shown). A deformable capsule may also be provided on the center top (not shown) and over a back neck portion of the protective gear (not shown). The positions of the deformable capsules are configured to the purpose of the wearable item (helmet, body armour, shoe soles . . . etc) depending on the type of activity and typical shocks that are received for which the wearable item is intended.

The pressure sensors 28 to which the deformable capsules are fluidically linked may be in the form of individual fluidic pressure sensors, or in the form of a common pressure sensor connected via valves to the fluidic connections of the individual deformable capsules. Fluidic pressure sensors are per se well known and a variety of different types of pressure sensors may be implemented in the scope of the present invention like a single input absolute or sealed type or a two input vented gauged relative pressure sensors usually with piezo resistive or capacitive type of pressure to electrical signal transduction.

The pressure sensors 28 may be connected to multiplexer 10, for instance an analog multiplexer of the electronic control system 8.

The electronic control system 8 comprises a computing system (micro computer) 9 including a sensor signal processing circuit 38 (sensor fusion engine), an onboard measurement signal analysis module 40 and a memory 46 including at least a data logger for recording sensor measurement information.

The electronic control system 8 may further comprise a communications system 11 configured for wireless communication of data to an external computing unit (herein called the base unit 100). The communications system 11 may further comprise a connector (not shown) for wired connection to an external computing unit and/or a power supply.

The electronic control system may further comprise a user interface 12 onboard the wearable item which may include for instance an audio system, for instance to issue an audible alarm in case of an impact that exceeds a certain threshold to alert the person or persons in the vicinity of an impact that requires attention. Other interface elements such as a display, lights, haptic outputs, and buzzers may also be provided.

The electronic control system further comprises an autonomous power source including for instance a battery. The electronic control system 8 may further comprise an energy capture interface, for instance an electrical connector or a wireless interconnection system such as an inductive power coupling for charging the internal battery. Energy may also be supplied to the electronic control system via a harvesting system such as systems based on the movement of the wearable item or by photovoltaic or thermal energy systems based on the heat difference between the wearer's body and the external environment. Such energy harvesting systems are per se known and need not be further described herein.

The sensor signal processing circuit 38 comprises input circuits for the sensors and a fusion module 38 for combining sensor signals whereby a plurality of impact sensing systems may be provided that work together as will be described in more detail hereinafter.

The onboard electronic control system 8 may advantageously further comprise an onboard measurement signal analysis module 40 that may include a machine learning module 42 and an impact analysis module 44 that receive the sensor signals from the sensor signal processing circuit and perform calculations of the signals to determine parameters that are relevant for the assessment of impacts on a person.

The measurement signal analysis module may be also externalized in the base station 100 whereby the measurement in the wearable item 2 performs only basic measurement functions sufficient to issue alarms for excessive impact strengths but not to perform a fine assessment of the history of multiple impacts. Nevertheless, in a preferred embodiment, the onboard measurement signal analysis module does provide an analysis of multiple impacts for greater security since the communication with an external device may not always function or always function well enough, especially depending on the environment in which the wearable item is being used and the activity.

The measurement system may comprise further sensors, for instance, environmental sensors such as temperature sensors and atmospheric pressure sensors, location sensors such as GPS tracking unit or other location sensors that may be used to track the wearer, and various other sensors such as a magnetometer, a sweat sensor, and an oximeter. Location information may be associated with impact event information to determine the position or location where the impact(s) took place. This may be used for instance to determine whether the impact(s) occurred with a fixed structure or with another subject or a player.

In addition to the measurement of impacts, the fluidic pressure sensing system 5 may advantageously be used to adjust the form and position of the sensor support structure and material aligning the person's body part to ensure a correct fit of the wearable item 2 to the person's body. This may for instance be used to ensure that any adjustment mechanisms such as fixing straps that hold the helmet or other body part to the person's head or other body part are correctly tensioned and adjusted. In effect, the proper fit and positioning of the protective gear is an important factor in improving protection and reducing the magnitude of an impact. For instance, a loosely placed helmet upon impact may cause greater injury than a well-fitting properly tensioned and positioned helmet.

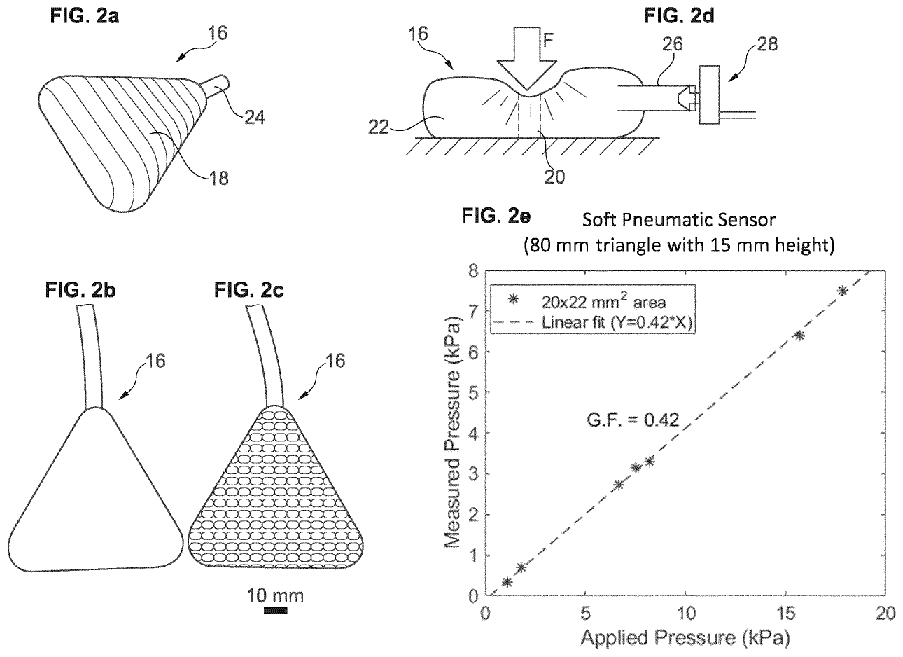

As illustrated in FIGS. 2a to 2c, in an embodiment of the invention, the deformable capsules are provided as triangular shaped flat capsules with dimensions of around 50 to 100 mm for the triangular sides and 5 to 20 mm in height, coupled at one of their apexes to a fluidic connection conduit 26. Other shapes may be provided within the scope of the invention such as rectangular, square, circular, oval, polygonal or irregular shapes that conform to the areas of the protective gear surfaces of which should be covered with the fluidic pressure sensing system.

An important advantage of the deformable capsules 16 is their ability to be easily integrated within typical materials used for protective padding within the protective gear. Moreover, the deformable capsules may participate in providing padding support and protection upon impact. Such construction is also light weight and easily conformable to the desired shape of the protective wearable gear.

Another important advantage of the fluidic pressure sensing system 5 according to an aspect of the present invention is the measurement sensitivity, whereby small deformations lead to pressure changes that are easily measurable by typical pneumatic pressure sensors thus leading to very accurate impact force measurement in an economical, lightweight and compact arrangement.

Fine measurement by the fluidic pressure sensing system 5 may advantageously be used inter alta to ensure proper fitting of the wearable item on the persons body part.

The impact sensing system further comprises an inertial measurement unit 7 configured for linear and angular acceleration of the wearable item. Various inertial measurement units 7 are per se well known in the art and typically comprise 3-dimensional accelerometers and/or gyroscopes. The inertial measurement unit may advantageously be used in conjunction with the fluidic pressure sensing system to compute a plurality of parameters useful for the assessment of an impact including the amplitude and direction of the impact force, the location of the impact on the wearable item 2, and the rotational and linear acceleration values.

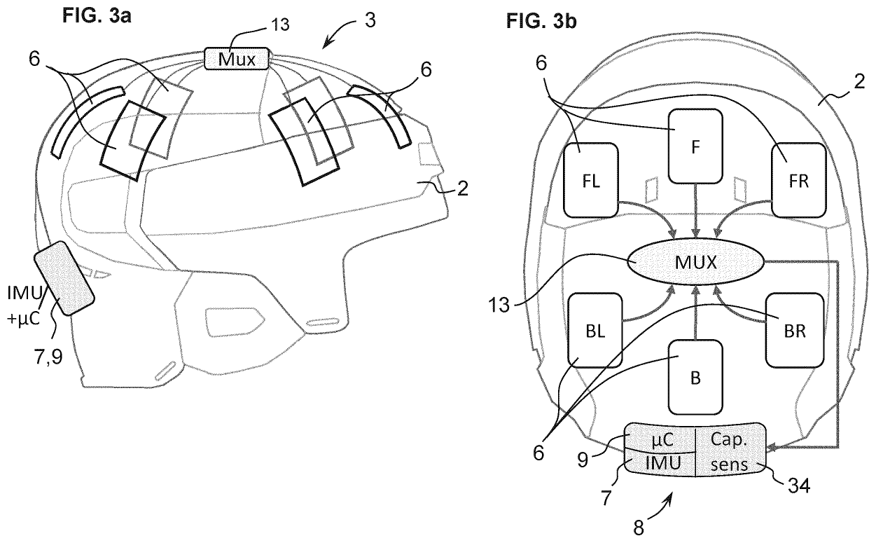

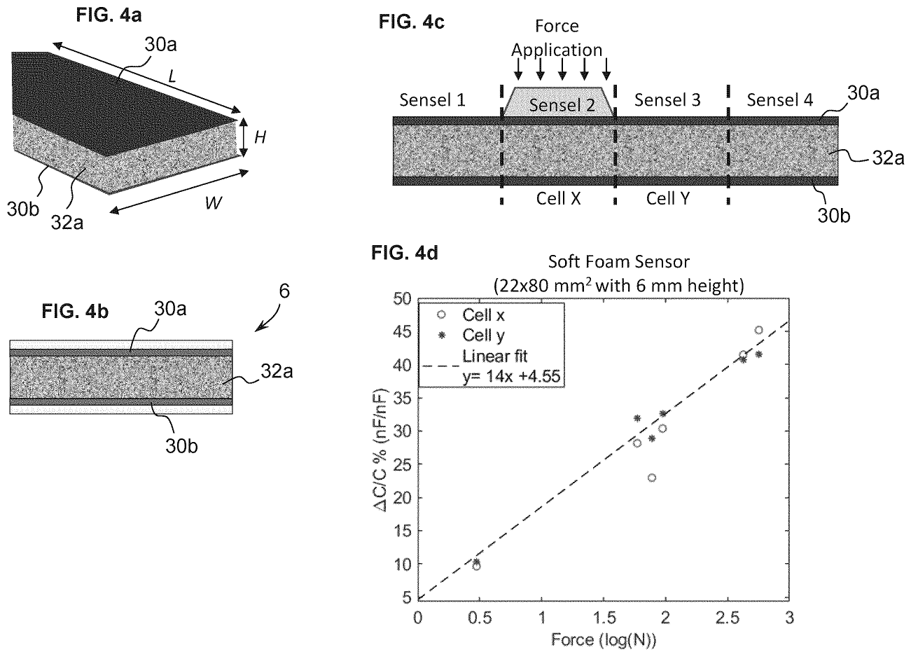

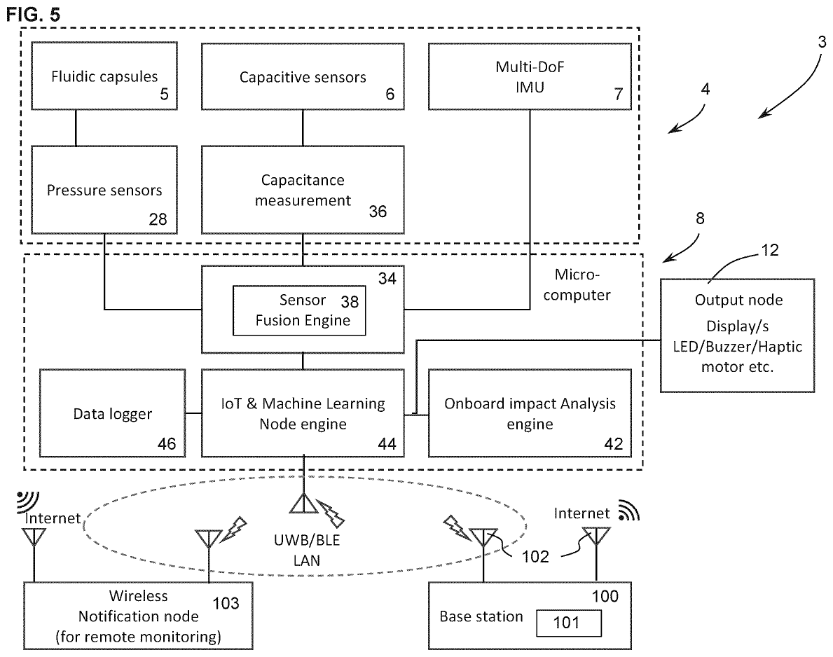

Referring to FIGS. 3a to 4d, according to another aspect of the invention, the impact sensing system may advantageously comprise a capacitive pressure sensing system, for instance advantageously in the form of capacitive sensors 6 in addition, or instead of the fluidic pressure sensing system. The capacitive sensors 6 comprise at least one deformable insulator layer 32a, 32b and at least a first and second conductor layer 30a, 30b, 30c on opposed sides of the deformable insulator layer 32a, 32b. Application of a force (pressure) on the capacitive sensor causes the deformable insulator layer to compress, reducing the distance between the conductive layers 30a, 30b and thus changing the capacitance value measured between the electrodes. The general principle of a capacitive sensor in a wearable item for measuring impact forces is per se known.

In an embodiment of the present invention, the capacitive sensors may be used in combination with the fluidic pressure sensors to improve the determination of the impact force, impact direction, and location of the impact. The deformable insulator layer may advantageously be in the form of a polymer foam, for instance, thermoplastics or thermosetting resins including polyethylene, nylon, TPU, TPE-U, epoxies, natural rubber, silicone or polyvinyl chloride (PVC) and may advantageously be integrated into a wearable item along with the fluidic pressure sensors, the capacitive sensors with deformable insulator layer also providing protective padding against an impact.

In a variant, the deformable capsules for the fluidic pressure sensing system may be formed within the deformable insulator layer of the capacitive sensors 6 such that the impact sensing system comprises an integrated combined fluidic pressure sensing system and capacitive sensing system.

Similarly to the fluidic pressure sensing system described previously, the capacitive sensing system 6 may comprise a plurality of individual capacitive sensing pads distributed on the sensor support structure 14 of the wearable item 2 at selected locations configured for optimal impact force and location measurement. For instance as illustrated in FIG. 3b showing a helmet, capacitive sensors may be placed on the front left (FL), front right (FR), back left (BL), back right (BR) and optionally front center (F), back center (B) and possibly in other positions such as right center, left center and center top. More or less capacitive sensors may be positioned. It may be further noted that with a capacitive sensor there may be a plurality of conductive electrodes on one side of the deformable insulator layer and a lesser number of electrodes on the other layer, for instance representing the reference or ground electrode.

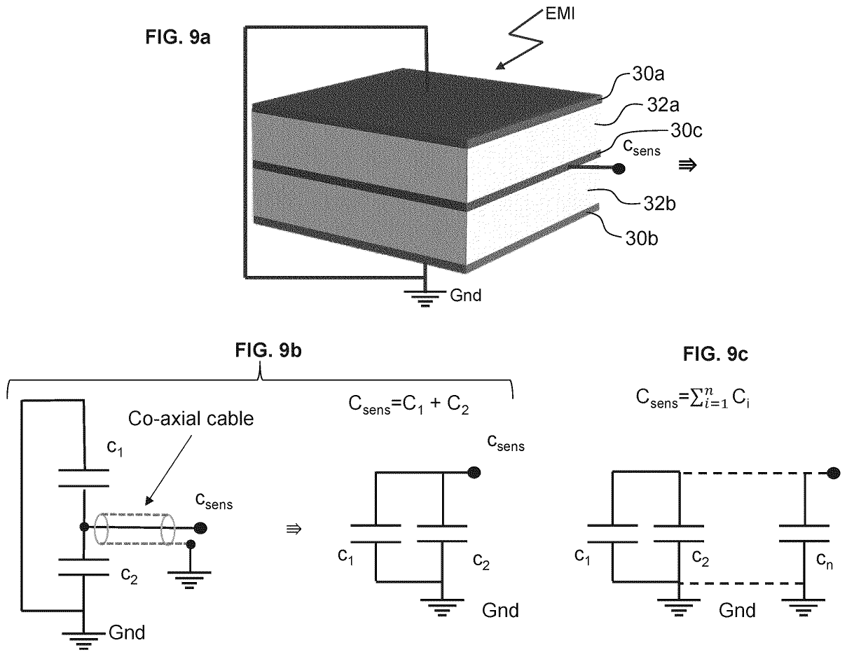

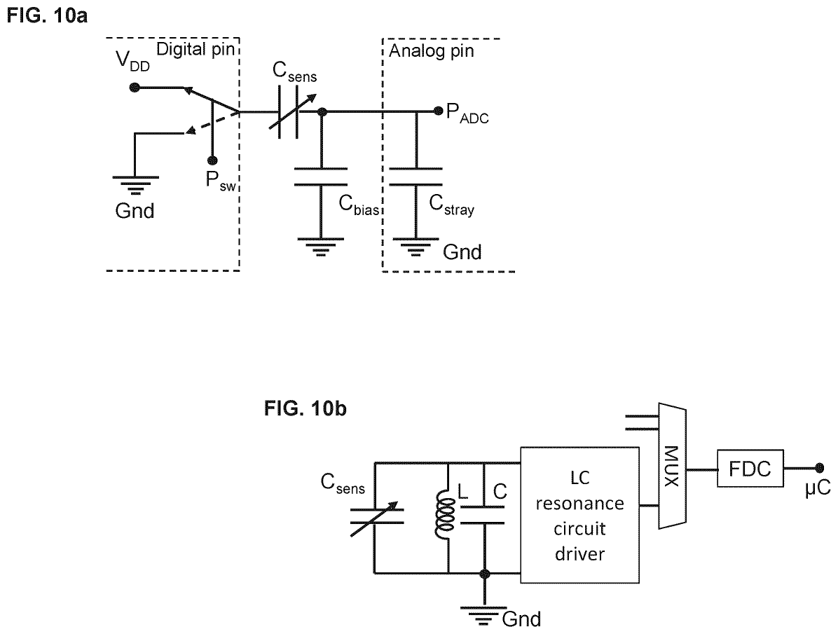

In an aspect of the invention, the capacitive sensor comprises two deformable insulator layers 32a, 32b separated by an electrode 32c, as illustrated in FIGS. 9a to 9c. The three electrodes may be connected together to obtain an electrically equivalent parallel configuration as illustrated in FIGS. 9b and 9c, with the aim of providing an additive capacitance value (C1+C2) where C1 and C2 are corresponding capacitance related to the deformation of the first and second deformable layers respectively. In this regard, the deformable layers may be provided with different elastic moduli such that the elastic strain of the two deformable insulator layers are different for a given force. The softer elastic layer would be adapted for a measurement of low impact forces and the stiffer deformable insulator layer with a higher elastic modulus adapted for measuring higher impact forces. The different elastic moduli may be achieved for instance by providing different materials, such as foams with different compositions or densities or porosity, or the same material with different forms or geometrical structures, or combinations of the same or different materials and geometrical structures. Alternatively or in addition, the first and second deformable layers may have different thicknesses such that the variation of the capacitance value C1 is different from the variation of the capacitance value C2 upon compression of the first and second layers due to impact or pressure applied on the capacitive sensor. Greater accuracy over the range of measurement of pressure and location thereof may thus be measured with this arrangement. It may be noted that this may also help for the fine adjustment of placement of the wearable item on a body for a better fit as already discussed above with regard to the fluidic pressure sensing system.

The measurement of impact forces, impact location and impact direction with the pressure sensors may be used in conjunction with the inertial measurement unit to compute the forces applied on the center of gravity of a person's head. Measurement of the linear acceleration and angular acceleration correlated with the impact location and magnitude allow to assess the effects of different types of impacts and the position of those impacts on the risk of injury. Measurement values may be stored and a history thereof used with machine learning techniques and medical outcomes to determine the risks associated with impacts depending on the nature of the impact(s) (position, force and frequency). The measurement system also allows to ensure a proper fit of the protective gear on a person thus reducing the risk of injury.

### Experimental Results

### Capacitive Foam Sensor Prototype:

A three-layer soft capacitive sensor strip was fabricated using a 5 mm thick silicone foam (Sil16, Silex Silicone Ltd., UK) and two 250 μm thick conductive silicone layers each. The sensor strip is 22×80 mm² that has a total capacitance of 13.5 pF±1 pF at 100 Hz and is dived into 4×1 sensor cells (sensel). The 4×1 sensels are 20 mm in width each, where the sensor performance of sensel-2 and sensel-3 is measured for different applied forces (FIG. 4c). We used known weights of 100 g, 600 g, 1600 g, and two random weights of 675 g, 736 g, 1411 (675+736) g for the capacitive sensor strip, and capacitance is measured at the initial points (FIG. 4d). A linear relationship between applied force and changed capacitance is observed on the logarithmic x-axis scale vs linear y-axis scale, due to the nonlinear compressive behavior of silicone foam. This property allows for high sensitivity at lower pressures ideal for tuning helmet fit (0.5-20 kPa) and then having a low sensitivity at higher compression to detect heavy impacts (>10 g). The foam properties like air bubble cell dimensions, silicone stiffness, and thickness of foam may be tuned as per the required range of senses. Further, a combination of different compressive stiffness foam layers can be used to have a tuned compression response for a specific application range.

### Soft Fluidic (Pneumatic) Capsule Prototype:

A 80 mm equilateral triangular soft pneumatic capsule, with a 10 mm height was fabricated using rapid liquid print (RLP) 3D printing method to have a monolithic form factor with a 4 mm diameter tubing outlet (FIGS. 2a-2c). This soft pneumatic sensor prototype was tested with the same weights and same size of wooden block with 20×22 mm² area to ensure similar applied pressure as in the above test for the capacitive sensors. A linear relationship between applied pressure vs measured pressure at the end of inlet tubing is observed (FIG. 2e). A part of applied forces is absorbed by the elastic silicone material that yields a gauge factor of 0.42 instead of −1. Beyond the proof-of-concept design, the RLP 3D printing method allows having complex 3D shapes that can be tuned for a specific purpose. For example, FIG. 2c shows a pillar-supported design with the same 80 mm side and 10 mm height triangular cell with higher stiffness and more like a foam to absorb more energy. A capsule can function as a foam with enclosed sides that not only help absorb the impact and regain the shape back elastically after the removal of forces but also measures accurately the changes in applied pressure without letting the complex shapes affect the measurements. The sensitivity of the sensors may be tuned through an iterative design process to compensate for the silicone to fluid ratio.

### Measurement System and Method

One of the aspects of the invention is to enable accurate prediction of impact location and the direction of the vector. FIG. 5 shows three sensor systems—fluidic 5, capacitive 6, inertial 7—that independently collect information on applied external linear and/or rotational forces and provide input to the sensor fusion engine 38.

The applied pressure on the fluidic capsules 5 may be measured through MEMS-based pressure sensors 28 which are highly accurate. These sensors 28 may be connected to the sensor fusion engine 38 through an analogue multiplexer 10.

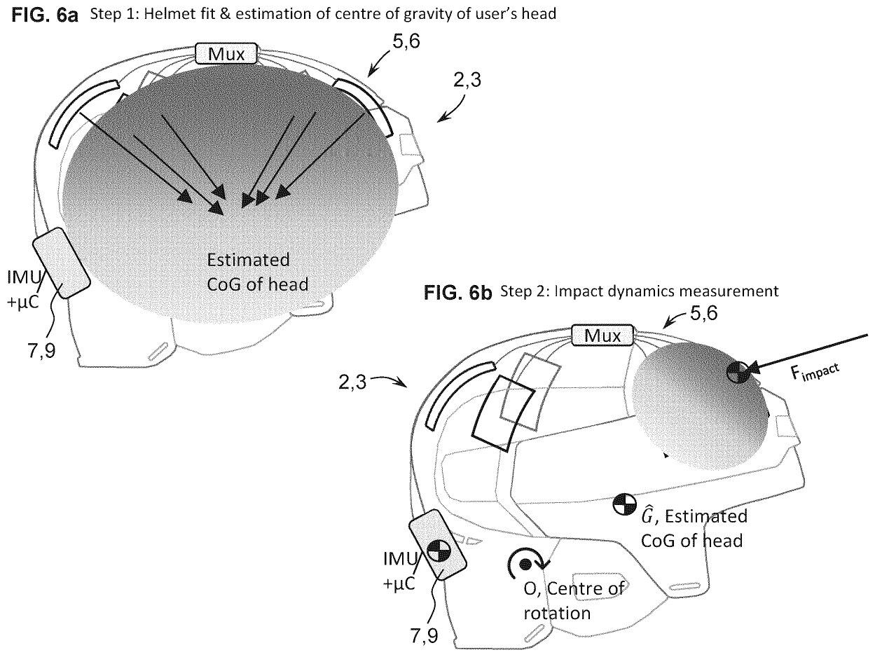

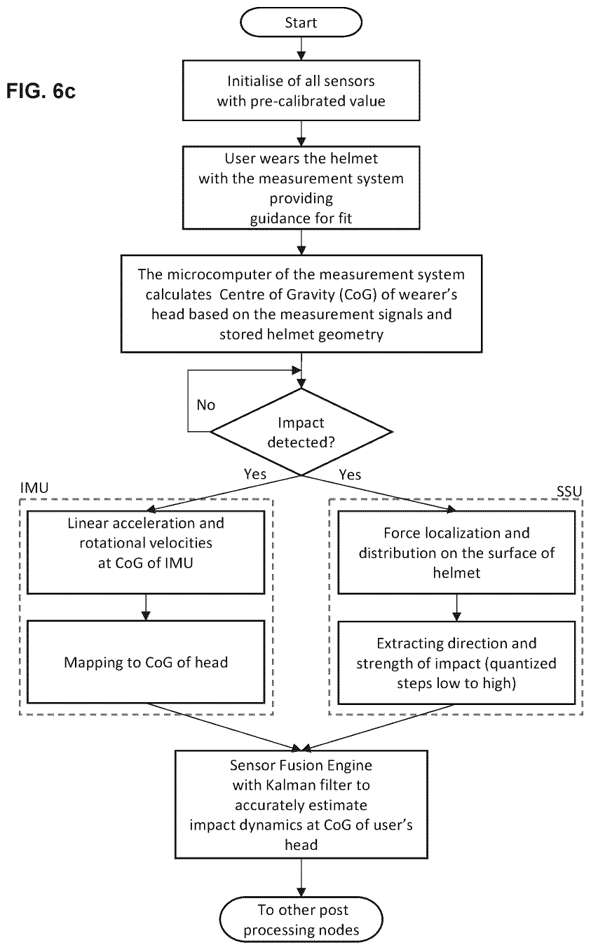

The plurality of distributed capacitive sensors 6 form an array connected to a capacitance measurement circuit 36 that measures the individual capacitance changes and feeds the information to the sensor fusion engine 38. Finally, a multi-degree of freedom inertial measurement unit (e.g. with accelerometers and a gyroscope) and optionally other sensors such as a barometer, and temperature sensors, combines the impact data and provides the information to the sensor fusion engine 38. The sensor fusion engine has the geometric information of the helmet and estimated location of the user's head and its centre of gravity as described later in relation to FIGS. 6a, 6b. The geometric information and measured sensor values from the pressure sensing system and inertial measurement unit are used to fuse all the data to obtain the best-guessed estimate of the applied forces during an external impact. The data may be stored locally in the memory 46 and transmitted wirelessly to an external storage system, including for instance a cloud-based storage system. The onboard data analytics engine 42 uses local processing to provide recurrent updates without requiring any external inputs.

The wireless notification node 103 may for instance comprise an external application installed on a monitoring person's (e.g. a coach of a sportsperson) smartphone or smartwatch receiving real-time notification in the case of severe impact detection requiring the attention of the monitoring person.

The base station unit 100 may be used to monitor the status of sensing nodes, for communication and synchronization purposes.

### Method for Helmet Fit and Impact Vector Detection:

The method of detection of the impact vector may be divided into two steps:
1. To ensure proper helmet fit, estimation of the centre of gravity (CoG) and centre of rotation (CoR) of the head of the wearer's head and,
2. Pressure sensors and inertial measurement unit sensor fusion-based impact vector estimation of the impact force.

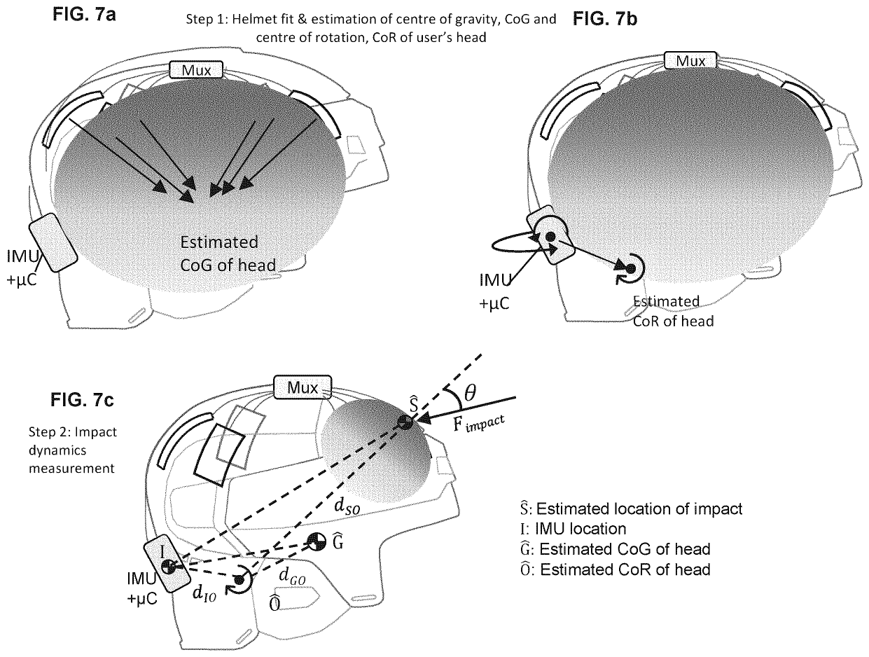

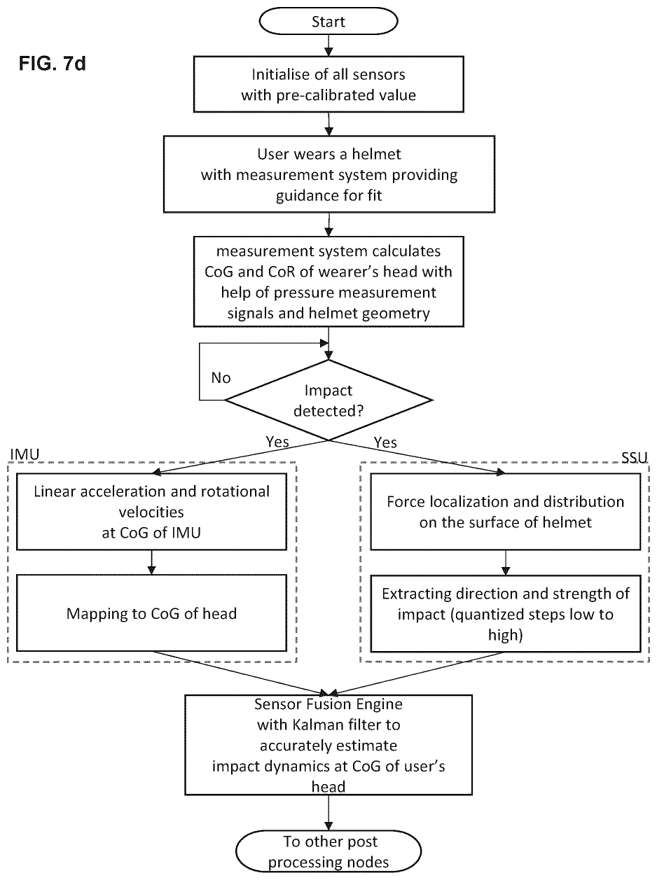

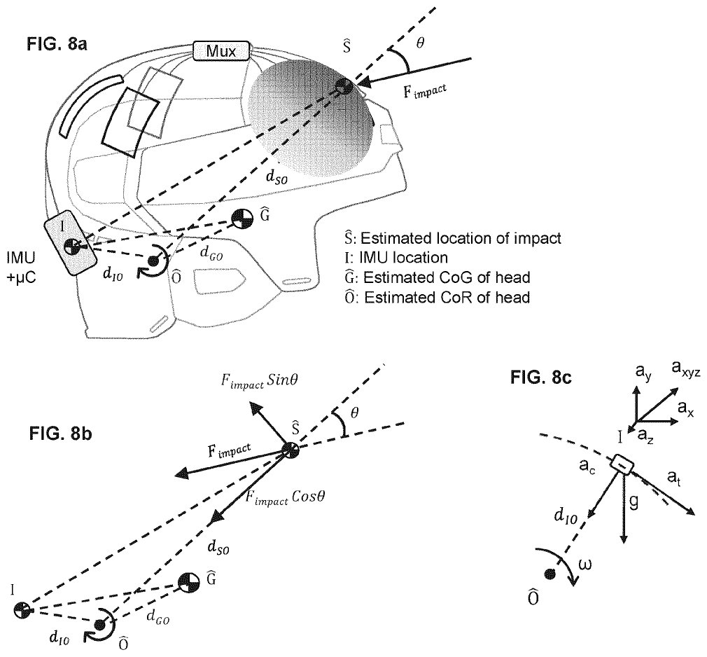

Step 1: Helmet fit and calibration: As illustrated in FIGS. 6a-6c, and 7a-7d all sensors are first initialized to pre-calibrated sensor values through their respective characterization and calibration procedures. The user is then guided using the pressure sensing system to ensure an optimal helmet through a follow-up procedure or visual aid from an application installed for instance online or locally on a portable computing device such as a smartphone, smartpad, or portable computer. The pressure points on the pressure sensing system during the helmet's fit and other markers, like helmet geometry and biomechanics of the human head are then used to estimate the centre of gravity of the user's head. The equation for the estimated centre of gravity can be given as:

$$\hat{G} ( x , y , z ) = {α}_{1} f ( {\sum }_{i = 1}^{n} {β}_{i} {F}_{i} ( x , y , z ) ) + {α}_{2} G ( x , y , z ) \qquad ( 1 )$$

where, f(x, y, z) is a linear combination of measured forces Fᵢ per soft sensor using the pressure sensing system,
G(x, y, z) is the initial seed location determined based on helmet geometry, and biomechanics of the human head, α* and βᵢ are numeric constants and n is the total number of pressure sensors.

Similarly, the centre of rotation, O, is estimated using the geometric placement of inertial measurement unit with respect to the helmet, biomechanics of standard human head-form and calibration procedure that translates the rotational kinematics from frame of reference of inertial measurement unit to the centre of rotation. The estimation function can be given as:

$$\hat{O} ( x , y , z ) = {γ}_{1} m ( tr ( {ω}_{I} ( x , y , z ) ) , {ω}_{i} ( x , y , z ) ) + {γ}_{2} O ( x , y , z ) \qquad ( 2 )$$

where, w_{I}(x, y, z) is rotational kinematics matrix with the frame of reference as the inertial measurement unit;
tr(·) is a mapping function/matrix to transfer rotational kinematics from the frame of reference of the inertial measurement unit to the centre of rotation, m(·) is a mapping function for obtaining the centre of rotation from the translation function/matrix, tr(·);
O(x, y, z) is initial seed location of the frame of reference of the centre of rotation based on the biomechanics of the human head and helmet geometry. γ₁ and γ₂ are filter coefficients tuned based on the covariance of estimation noise.

After the guided helmet fit process using the pressure sensing system, as a part of the calibration procedure for the inertial measurement unit, the wearer may then be asked to move the head left, right, up, down, and circular fashion to update the mapping function (tr(w_{I})) for improved prediction of the centre of rotation (CoR).

Step 2: Following the CoR and CoG estimation step, the onboard microcomputer is ready for the impact analysis step. Upon an impact event, the system triggers collection of the data from the pressure sensing system and inertial measurement unit and the data are then filtered and combined through the sensor fusion engine to obtain the location, strength, and direction of the impact vector with respect to both the helmet and CoG of user's head. The collective dynamics information may be made available for other functional nodes for post-processing.

The pressure sensing system uses a distributed sensing network to localize the impact region based on collective force data (|Fᵢ|, i=1:n) from all the sensors shown as Ŝ(x, y, z) in FIGS. 8a to 8c. FIGS. 8a to 8c illustrate the free body diagram of the impact force application (F_{impact}) and corresponding rotational and translational components whereby the amplitude of impact force (|F_{impact}|) and direction (θ) are estimated using pressure sensing system and inertial measurement unit sensor fusion. The force responsible for the rotation of the head is perpendicular to the distance dₛₒ, where Ô is the centre of rotation previously estimated using biomechanics and helmet size. The rotation velocity (ω ô) at point Ô is translated from measured angular rotation (ω_{I}). The location for inertial measurement unit placement is known and the centre of gravity (CoG) and centre of rotation (CoR) are estimated previously using equation (1) and (2).

Torque is generated due to incident impact force, F_{impact}, at the CoR which also leads to angular velocity of ω ô. The component of F_{impact} responsible for torque can be equated to the angular velocity and moment of inertia (M.I.) product to obtain the angle of incenting impact force with respect to the centre of rotation CoR. Hence, the incident angle (θ) part of the impact force vector can be measured as:

$$θ = {θ}_{( SSU )} = {sin}^{- 1} ( \frac{M . I . \cdot {ω}_{\hat{O}}^{.}}{\vert {F}_{impact} \vert \cdot {d}_{SO}} ) ; \qquad ( 3 )$$

The impact force incident angle part can also be measured using the translational acceleration, a_{G}=[a_{xG}, a_{yG}, a_{zG}]^{T} (say) from the inertial measurement unit (IMU) translated to the centre of gravity point as follows:

$$θ = {θ}_{( IMU )} = {[ {θ}_{xy} {θ}_{yz} {θ}_{xz} ]}^{T} = [ {tan}^{- 1} ( \frac{{a}_{y}}{{a}_{x}} ) {tan}^{- 1} ( \frac{{a}_{z}}{{a}_{y}} ) {tan}^{- 1} ( \frac{{a}_{x}}{{a}_{z}} ) ] ; \qquad ( 4 )$$

Finally, the measured incident force angles from the pressure sensing system and inertial measurement unit obtained from equations (3) and (4) may be fused together using a Kalman filter.

Along with the location, direction and magnitude of incident impact force, the linear acceleration at the CoG, a_{G} is crucial to analyze the severity of the impact. The linear acceleration (a_{I} or a_{G}) can be divided into four components: translational acceleration (a_{xyz}), acceleration due to gravity (g), centripetal acceleration (a_{c}) and tangential acceleration (aₜ) at the point of reference I or Ĝ in our case, as shown in FIGS. 8a to 8c, and equations (5) and (6) below:

At the frame of reference of inertial measurement unit, point I:

$${a}_{I} = {a}_{xyzI} + g + {a}_{cI} + {a}_{tI} ; \qquad ( 5 )$$

$${a}_{tI} = {\dot{ω}}_{I} \cdot {d}_{IO} ; {a}_{cI} = {ω}_{I}^{2} \cdot {d}_{IO} ;$$

At CoG, point Ĝ:

$${a}_{G} = {a}_{xyzI} + g + {a}_{cG} + {a}_{tG} ; \qquad ( 6 )$$

$${a}_{tG} = {\dot{ω}}_{I} \cdot {d}_{GO} ; {a}_{cG} = {ω}_{I}^{2} \cdot {d}_{GO} ;$$

$$Now ,$$

$${a}_{xyzG} = {a}_{xyzI} \qquad ( 7 )$$

$$∴ {a}_{G} = {a}_{G ( IMU )} = {a}_{I} - {\dot{ω}}_{I} \cdot {d}_{IO} - {ω}_{I}^{2} \cdot {d}_{IO} + {ω}_{I}^{2} \cdot {d}_{GO} + {\dot{ω}}_{I} \cdot {d}_{GO} + g ;$$

The values of ω_{I} and αᵢ are obtained from the inertial measurement unit and can be used to calculate a_{G}. Similarly, as can be seen in FIG. 7, assuming, mass ‘m’ for the helmet and wearer's head as a rigid body, a_{G} can be calculated as F_{impact} Cosθ/m. The impact acceleration measured by the pressure sensing system has to be compensated for the shock absorption at the neck-head muscles to realistically represent the translational acceleration, a_{G}; the resulting equation can be given as:

$${a}_{G} = {a}_{G ( SSU )} - ϕ = {F}_{impact} cos θ / m - ϕ ; \qquad ( 8 )$$

Where φ, is a matrix depending on the wearer's biomechanics at the neck and head estimated independently.

A Kalman filter-based sensor fusion approach can be used to predict a more accurate translational acceleration (â_{G}) at the CoG by combining the a_{G(IMU)} and a_{G(SSU)} measurements from equation (7) and (8), as:

$${\hat{a}}_{G \vert k + 1} = KF ( {a}_{G \vert k ( SSU )} - {ϕ}_{k} , {a}_{G \vert k ( IMU )} , {Q}_{( IMU ) ,} {Q}_{( SSU )} ) . \qquad ( 9 )$$

where, KF(·) is a Kalman filter estimator for multiple sensors with different variance[24]. k, k+1 are the subsequent discrete measurement instances in time. Q_{(IMU)}, and Q_{(SSU)} are corresponding covariance matrices for the inertial measurement unit and pressure sensing system.

### LIST OF FEATURE REFERENCES

Body impact evaluation system
   Base station 100
      Computing system 101
         Impact measurement module
      Communications system 102
         Wireless node
   Wearable item 2
      Helmet
         Sensor support structure 14
      Footwear
   Measurement system 3
      Impact sensing system 4
         Fluidic pressure sensing system 5
         Deformable capsule 16
         Capsule outer wall 18
         Internal support 20
         Chamber 22
         Inlet/outlet 24
         Fluidic connection conduits 26
         Pressure sensor 28
         Capacitive sensors 6
         Conductor layers 30a, 30b, 30c
         Deformable insulator layer(s) 32a, 32b
         Polymer foam
         Capacitance measurement output circuit 36
         Inertial measurement unit 7
         Accelerometers
         Gyroscope
      Environmental sensors
         Temperature sensor
         Atmospheric pressure sensor (barometer)
      Magnetometer
      Sweat sensor
      Oximeter
      Location sensors
         GPS
      Electronic Control system 8
         Computing system (Microcomputer) 9
         Sensor signal processing circuit 34
         Sensor signal fusion module 38
         Onboard measurement signal analysis module 40
         Machine learning module 42
         Impact analysis module 44
         Memory 46
         (including data logger)
         Multiplexer 10 for fluidic pressure sensors
         Analog multiplexer
         Multiplexer 13 for capacitive sensors
         Communications system 11
         User interface 12
         (Audio, Display, Lights, Haptic output, buzzer)

1. A measurement system for a wearable item comprising an electronic control system and an impact sensing system, the impact sensing system including a capacitive pressure sensing system comprising a plurality of capacitive sensors, the capacitive sensors comprising a deformable insulator layer and at least first and second electrodes on opposed sides of the deformable insulator layer, the capacitive sensors arranged in a distributed manner over a surface for covering a body part subject to impact, characterized in that the capacitive sensors comprise first and second deformable insulator layers separated by a third electrode, the first insulator layer having an elastic modulus that is different to an elastic modulus of the second insulator layer, or the first insulator layer having a thickness that is different to a thickness of the second insulator layer, the capacitive sensors configured to measure a pressure applied on a major surface of the sensor transverse to the stacking direction of the electrodes and deformable insulator layers.

2. The measurement system according to claim 1 wherein the deformable insulator layers comprise or consist of a polymer open or closed cell foam material.

3. The measurement system according to claim 1 wherein the electronic control system further comprises an onboard measurement signal analysis module and a memory, the onboard measurement signal analysis module including a machine learning module and an impact analysis module configured to compute at least linear acceleration and inertial angular acceleration on a center of gravity and a center of rotation of a body part.

4. The measurement system according to claim 1 comprising a multiplexer connected to the capacitive pressure sensing system, the multiplexer comprising an analog multiplexer with or without a digital converter to aid in preventing sensor signal from corrupting due to noise arranged between sensing elements of the fluidic and/or capacitive pressure sensing system and separate from the computing system.

5. The measurement system according to claim 2 further comprising a wireless communications system including any one or more of a Bluetooth communication system, a Wifi communication system, a GSM/LTE communication system, or a near field communication system for connection to an external computing unit.

6. (canceled)

7. (canceled)

8. (canceled)

9. (canceled)

10. A wearable item comprising a sensor support structure and the measurement system according to claim 1 mounted on or in the sensor support structure.

11. The wearable item according to claim 10 wherein the wearable item is a protective head gear, for instance in the form of a helmet.

12. (canceled)

13. (canceled)

14. (canceled)

15. (canceled)

16. A measurement system for a wearable item comprising an electronic control system, an inertial measurement unit, and an impact sensing system, the impact sensing system including a fluidic pressure sensing system comprising a plurality of capsule chambers each filled with a fluid, and pressure sensors connected to the capsule chambers configured to measure a variation in volume due to pressure applied on an outer wall of the chamber, the electronic control system receiving measurement signals from the pressure sensors and inertial measurement unit, the electronic control system configured for processing said measurement signals to compute parameters related to an impact.

17. Measurement system according to claim 16 wherein the chambers are formed within a deformable polymeric material.

18. Measurement system according to claim 17 wherein the capsule chamber is formed within a deformable capsule separately formed from a support structure of the wearable item, the deformable capsule comprising a capsule outer wall, a plurality of deformable capsules arranged in a distributed manner for covering a body part subject to impact, each deformable capsule connected individually to a pressure sensor or an individual inlet of a common pressure sensor.

19. Measurement system according to claim 16 wherein the capsule chamber comprises a surface area of between 3 cm² to 300 cm², preferably in a range of 10 cm² to 200 cm² over a surface that is substantially orthogonal to an impact direction, and a thickness in said impact direction in a range between 5 and 35 mm, preferably in a range of between 8 and 20 mm.

20. Measurement system according to claim 16 wherein the impact sensing system further comprises a capacitive sensing system including a plurality of capacitive sensors comprising a deformable insulator layer and at least first and second electrodes on opposed sides of the deformable insulator layer, the capacitive sensors arranged in a distributed manner over a surface for covering a body part subject to impact.

21. Measurement system according to claim 16 wherein the electronic control system comprises a sensor signal processing circuit (34) configured to process both the inputs for the inertial measurement unit and the pressure sensors, and optionally from additional sensors such as the capacitive sensors, for generating impact measurement parameters including an amplitude of the impact force, a location of the impact force and optionally a direction of the impact force.

22. Measurement system according to claim 16 wherein the electronic control system further comprises an onboard measurement signal analysis module and a memory, the onboard measurement signal analysis module including a machine learning module and an impact analysis module configured to compute at least linear acceleration and inertial angular acceleration on a center of gravity and a center of rotation of a body part.

23. Measurement system according to claim 16 comprising a multiplexer connected to the fluidic pressure sensing system, the multiplexer comprising an analog multiplexer arranged between sensing elements of the fluidic pressure sensing system and separate from the computing system.

24. Measurement system according to claim 16 further comprising a wireless communications system including any one or more of a Bluetooth communication system, a Wifi communication system, a GSM/LTE communication system, or a near field communication system for connection to an external computing unit.

25. A wearable item comprising a sensor support structure and the measurement system according to claim 16 mounted on or in the sensor support structure.

26. Wearable item according to claim 25 wherein the wearable item is in the form of a helmet, the sensor support structure is formed of a deformable polymeric self-supporting material, the fluidic pressure sensing system being integrated into the sensor support structure, optionally wherein the fluidic chambers of the measurement system are formed within the sensor support structure.

27. Wearable item according to claim 25 wherein the wearable item is in the form of a helmet, a multiplexer being positioned in a top region of the helmet, optionally wherein the multiplexer is a slave analogue or micro-controller based digital multiplexer configured to provide sensor data on demand to microcontroller master.

28. Wearable item according to claim 25 wherein the wearable item is in the form of a helmet, and wherein the chambers of the fluidic pressure sensing system include chambers positioned in the helmet at least on a front left, front right, back left and back right positions, optionally wherein the measurement system further includes chambers in a front center and back center position.
